(19) United States
(12) Patent Application Publication
(10) Pub. No.: US 2018/0243112 A1
(43) Pub. Date: Aug. 30, 2018

(54) INTERCHANGEABLE LOCAL INTERFACE PROSTHETIC SOCKET APPARATUS AND SYSTEM

(71) Applicant: Extremiti3D, LLC, Johns Island, SC (US)

(72) Inventor: Barry Hand, Johns Island, SC (US)

(21) Appl. No.: 15/905,545

(22) Filed: Feb. 26, 2018

Related U.S. Application Data
(60) Provisional application No. 62/463,789, filed on Feb. 27, 2017.

(51) Int. Cl. A61F 2/80

(52) U.S. Cl. CPC A61F 2/80; B33Y 80/00

(57) ABSTRACT

An additive manufacturing method and apparatus for interchangeable local interface support geometry and materials in lower limb prosthetic sockets. An interchangeable local interface prosthetic socket is configured to enable a user of the socket to configure variable support, pressure, and comfort characteristics of lower limb prosthetic sockets by selectively replacing an interface panel and/or distal cup with an alternative interface panel and/or distal cup being configured to have different physical characteristics.

### CROSS-REFERENCE TO RELATED APPLICATIONS

This application claims the benefit of U.S. Provisional Patent Application Ser. No. 62/463,789, filed on Feb. 27, 2017 entitled “METHOD AND APPARATUS FOR BESPOKE INTERFACE SUPPORT IN PROSTHETIC DEVICES”, the disclosure of which is hereby incorporated in its entirety at least by reference.

### FIELD

The present disclosure relates to the field of lower limb prosthetics and methods of manufacturing the same; in particular, an additive manufacturing method and apparatus for interchangeable local interface support geometry and materials in lower limb prosthetic sockets.

### BACKGROUND

There are approximately 84,500 to 114,000 new lower-limb amputations each year in the United States. Amputation rates are rising each year, in part because of the rapid increase in diabetes and also because of improvements in treating traumatic injury and vascular disease. More of the patients experiencing these problems are able to live longer but may require limb amputation in order to survive. Further, the recent wars in Iraq and Afghanistan have caused an increase in the number of servicemen and women who undergo an amputation, typically young individuals who are otherwise healthy. Because of the early age at which the amputation occurred, these individuals will be prosthesis (i.e. an externally applied device used to replace wholly, or in part, an absent or deficient limb segment) users for many years. Thus, there is a strong need to create quality prosthetic limbs for the increasing lower-limb amputee population.

The design of an effective prosthetic socket is crucial to the rehabilitation and overall health of a person with an amputated limb. This point cannot be overemphasized. Most of the time and energy a practitioner applies in making a prosthesis is spent on fabricating the socket that must be fitted to the residual limb. The prosthetic socket must be shaped so that it supports the residual limb in load tolerant areas, while avoiding irritation of sensitive regions on the limb that contact the inner surface of the socket. If these criteria are not achieved, residual limb soft tissue breakdown often occurs when the patient uses the prosthesis. The result of a poor socket fit may include painful sores, blisters, ulcers, or cysts on the residual limb that typically restrict continued prosthesis use and, in severe cases, necessitate a further amputation to a higher anatomical level which can lead to further disability. The incidence of skin breakdown in lower-limb amputees has been reported to be from 24% to 41%. Accordingly, at any one time, as many as 41% of prosthesis users may be experiencing breakdown of the tissue on the residual limb. The principal cause of such breakdown is a poorly fitting prosthetic socket.

In recent years, manufacturers of prosthetics have turned to 3D printing technologies to reduce the cost of manufacturing and provide better fitting prosthetics through the use of computer-assisted scans of a patient's residual limb in order to build better fitting sockets. However, even a well-fitted prosthetic socket does not always provide the optimal fit or performance characteristics for every level of activity in which the user may choose to engage. Specifically, a user of a lower limb prosthetic socket may prefer a different “fit” for comfort and performance purposes when wearing the lower limb prosthetic socket in the context of different activities. For example, when a user of a lower limb prosthetic socket is engaging in higher intensity physical activity (e.g., exercising, jogging, biking, etc.), the user may prefer a tighter fit of the lower limb prosthetic socket on his residual lower limb. Conversely, when a user of a lower limb prosthetic socket is engaging in lower intensity physical activity (e.g., working in an office environment, engaging in personal activities, etc.), the user may prefer a more relaxed fit of the lower limb prosthetic socket on his residual lower limb. Some users may have multiple sockets configured for this purpose. However, multiple sockets for multiple use cases is cost prohibitive for many users and may be impractical. What is needed, therefore, is a lower limb prosthetic socket that enables a user to selectively change the fit of a socket without needing to change the entire socket.

Through applied effort, ingenuity, and innovation, Applicant has identified a number of deficiencies and problems with interface support in lower limb prosthetic sockets. Applicant has developed a solution that is embodied by the present invention, which is described in detail below.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

An object of the present disclosure is a modular lower-limb prosthetic apparatus comprising a 3D printed socket comprising an interior portion and an exterior portion defining a socket wall therebetween, the socket wall extending from an upper perimeter, defining a proximal opening of the interior portion, to a lower perimeter defining a distal plane, the distal plane defining a circumference, the 3D printed socket being configured according to a digital scan of a residual lower limb such that the interior surface of the socket wall mimics a surface of the residual lower limb, the 3D printed socket being constructed from a plurality of stratified layers; a panel window being disposed on the 3D printed socket, the panel window comprising side walls extending from the interior portion of the 3D printed socket to the exterior portion of the 3D printed socket to define an aperture through the socket wall, the panel window having at least one panel interface portion being disposed on at least one of the side walls; a first interface panel being removably coupled to the panel window, the first interface panel having at least one panel window interface portion being removably engaged with the at least one panel interface portion; and, a first distal cup being removably coupled to a distal interface portion of the 3D printed socket, the first distal cup having an upper surface configured to mimic a distal surface of a residual limb of a user, and a lower surface configured to interface with the distal interface portion of the 3D printed socket.

Another object of the present disclosure is a variable interface lower-limb prosthetic socket apparatus comprising a 3D printed socket comprising an interior portion and an exterior portion defining a socket wall therebetween, the socket wall extending from an upper perimeter, defining a proximal opening of the interior portion, to a lower perimeter defining a distal plane, the distal plane defining a circumference, the 3D printed socket being configured according to a digital scan of a residual lower limb such that an interior surface of the interior portion mimics a surface of the residual lower limb, the 3D printed socket being constructed from a plurality of stratified layers; a panel window being disposed on the 3D printed socket, the panel window comprising side walls extending from the interior portion of the 3D printed socket to the exterior portion of the 3D printed socket to define an aperture through the socket wall, the panel window having at least one panel interface portion; at least one interface panel being removably coupled to the panel window via the least one panel interface portion, the at least one interface panel being operably configured to define one or more support, pressure, and comfort characteristics of the 3D printed socket; and, a distal cup being mateably coupled to a distal interface portion of the 3D printed socket such that the distal cup is configured to be selectively installed or removed from the distal interface portion of the 3D printed socket by the user, the distal cup having an upper surface configured to mimic a distal surface of a residual limb of a user, and a lower surface configured to interface with the distal interface portion of the 3D printed socket.

Yet another object of the present disclosure is a modular lower-limb prosthetic system for interchangeable local interface support, the modular lower-limb prosthetic system comprising a 3D printed socket comprising an interior portion and an exterior portion defining a socket wall therebetween, the socket wall extending from an upper perimeter, defining a proximal opening of the interior portion, to a lower perimeter defining a distal plane, the distal plane defining a circumference, the 3D printed socket being configured according to a digital scan of a residual lower limb such that the interior surface of the socket wall mimics a surface of the residual lower limb, the 3D printed socket being constructed from a plurality of stratified layers; a panel window being disposed on the 3D printed socket, the panel window comprising side walls extending from the interior portion of the 3D printed socket to the exterior portion of the 3D printed socket to define an aperture through the socket wall, the panel window having at least one panel interface portion being disposed on at least one of the side walls; a first interface panel being removably coupled to the panel window, the first interface panel having at least one panel window interface portion being removably engaged with the at least one panel interface portion; a first distal cup being removably coupled to a distal interface portion of the 3D printed socket, the first distal cup having an upper surface configured to mimic a distal surface of a residual limb of a user, and a lower surface configured to interface with the distal interface portion of the 3D printed socket; a second interface panel being configured to be removably coupled to the panel window in place of the first interface panel, the second interface panel having at least one panel window interface portion being removably engaged with the at least one panel interface portion; and, a second distal cup being configured to be removably coupled to a distal interface portion of the 3D printed socket in place of the first distal cup, the second distal cup having an upper surface configured to mimic a distal surface of a residual limb of a user, and a lower surface configured to interface with the distal interface portion of the 3D printed socket.

The foregoing has outlined rather broadly the more pertinent and important features of the present invention so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present invention. It should be realized by those skilled in the art that such equivalent structures do not depart from the spirit and scope of the invention as set forth in the appended claims.

### BRIEF DESCRIPTION OF DRAWINGS

The above and other objects, features and advantages of the present disclosure will be more apparent from the following detailed description taken in conjunction with the accompanying drawings, in which:

FIG. 1 is a perspective view of a prosthetic socket, according to an embodiment of the present disclosure;

FIG. 2 is a cross sectional view of a prosthetic socket, according to an embodiment of the present disclosure;

FIG. 3 is a process flow diagram of a method for changing local interface support geometry and materials within a prosthetic socket, according to an embodiment of the present disclosure;

FIG. 4 is a plan view of a prosthetic socket, according to an embodiment of the present disclosure;

FIG. 5 is a plan view of a prosthetic socket and cross sectional views of alternative attachment structures for an interchangeable panel of a prosthetic socket, according to an embodiment of the present disclosure;

FIG. 6 is a plan view of a prosthetic socket and cross sectional views of alternative surface structures for an interchangeable panel of a prosthetic socket, according to an embodiment of the present disclosure;

FIG. 7 is a plan view of a prosthetic socket, according to an embodiment of the present disclosure; and,

FIG. 8 is a plan view of a prosthetic socket and alternative cross sectional views of a distal cup of the prosthetic socket, according to an embodiment of the present disclosure.

### DETAILED DESCRIPTION

Exemplary embodiments are described herein to provide a detailed description of the present disclosure. Variations of these embodiments will be apparent to those of skill in the art. Moreover, certain terminology is used in the following description for convenience only and is not limiting. For example, the words “right,” “left,” “top,” “bottom,” “upper,” “lower,” “inner” and “outer” designate directions in the drawings to which reference is made. The word “a” is defined to mean “at least one.” The terminology includes the words above specifically mentioned, derivatives thereof, and words of similar import.

A prosthetic socket can be printed using a number of additive manufacturing (AM) methods. During the course of wearing a prosthetic socket, a user applies force and torque to the socket from the interior portion of the socket through contact with the residual limb. These forces may be created from the bone of the amputated limb pressing through soft tissue (e.g. muscle and skin) against the inside of the socket, for example. The location where the force is applied can often result in pain to the user, caused by bruising or abrasion of the user's skin against the liner. Methods for relieving this interface force include the application of liners made of gel, adding foam to the inside of the socket at the location of the force, changing the shape of the socket at the location to be cavernous to prevent or reduce contact, or carving holes or openings in the socket wall to prevent or reduce contact. These methods are effective in some cases. Each of these methods require knowledge and effort on behalf of the caregiver (i.e. Prosthetist), and include cutting, grinding and/or heating the prosthetic socket. In addition, several trial sockets may need to be manufactured to find the right solution for the user. Quite often, the creation of a method for relieving the forces causing the pain also eliminates reactive forces providing support for the limb during use. Supporting forces make the act of walking or otherwise using the artificial limb possible and more natural. In other cases, increasing interface pressure may be desired, in which case more material may be added in certain locations.

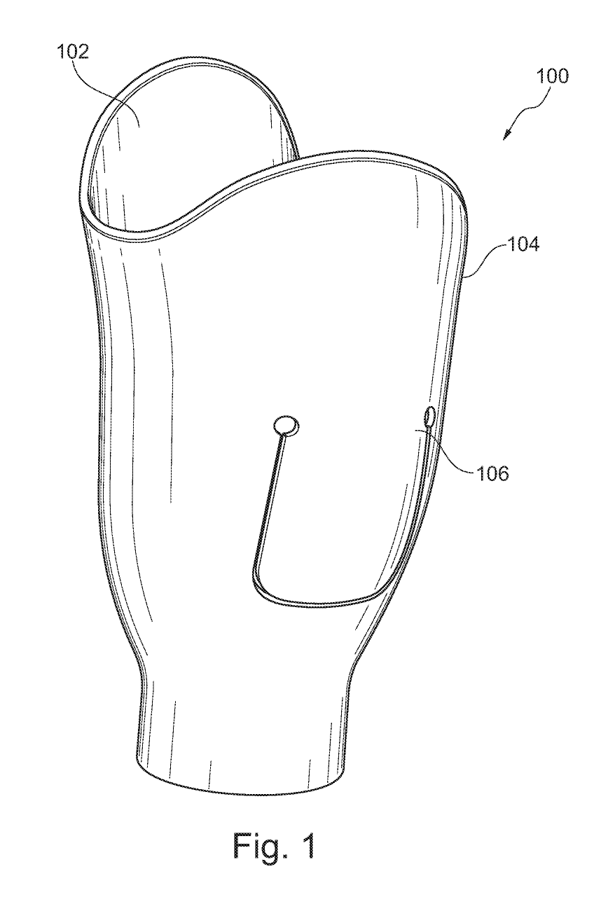

Referring now to FIG. 1 a perspective view of a prosthetic socket 100, according to an embodiment of the present disclosure, is shown. Embodiments of the present disclosure enable a method to tailor and manage the interface forces discussed above, while continuing to provide support. A prosthetic socket 100 is manufactured using a scanned computer model (CAD) of a limb or socket, into which openings 106 are designed into the wall 104 of the socket at the location where pain or discomfort is indicated. Prosthetic socket 100 has an interior portion 102, which is configured to receive and hold the target limb. Opening 106 can be traced onto the socket virtually, as identified by the prosthetist, and may be any shape or size. The location of the opening 106 can be predicted by the prosthetist at the time of evaluating and scanning the patient's limb. Using standard CAD techniques, there can also be a model created to exactly match the piece “removed” from the opening to fill the opening 106. This filler piece can be designed so that it may be placed in the opening with various possible methods for attachment. These methods include shapes creating a snap, holes for fasteners, Velcro®, apertures for cables, etc. The filler piece itself may be designed with many different features which allow for changing needs of the patient. For instance, more support may be desired for exercise, where less support is needed for continuous standing at a work station. The piece may have features such as a lever or leaf spring, which may be manufactured directly into the piece. A lever which is attached on one end to the filler piece is shaped to match the anatomy of the residual limb at the precise location of the force in question. This lever would be designed to flex away from the force at a predetermined rate (lbs/in) that would remove the painful force, but continue to provide support. The spring force of this lever may be changed to be high load or low load by simply changing the shape of the lever. Indeed, there may be multiple filler pieces that the patient may select to use for different activities. In the case of a location that sees repeated wear, a similar lever can be designed such that forces are relieved with minimal shear force on the skin while support is still provided. Other embodiments of this relief lever include multiple shapes with multiple attachment locations. The filler pieces can also be made of materials different from the socket, such as soft or elastomeric materials, clear materials for evaluation, or another material adhered to the filler piece.

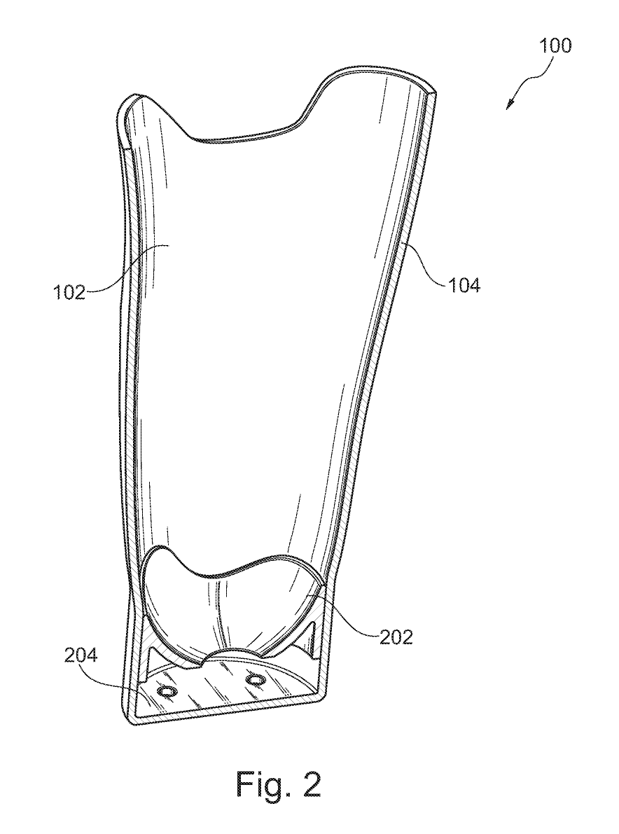

Referring now to FIG. 2, a cross-sectional view of prosthetic socket 100 is shown, according to an embodiment of the present disclosure. The methods for relieving the interface forces described above can add significant cost to a socket if the components are fabricated with traditional methods by hand. In an embodiment, the spring/lever shapes are fabricated using 3D printing as a contiguous part of the socket. The design of the spring is determined using processes known in the state of the art and outside the scope of this patent. Once determined, the spring/lever design is added to the 3D model of the socket in a computer aided design or other solid modeling software package. The spring/lever can be printed in any complex shape required at no additional cost compared to a socket printed without the spring/lever. In a preferred embodiment, a window opening is created in the socket at the location of the spring, and is designed to enable a part to be removably attached (snapped or fastened). The part which fills the window may be the spring/lever, or whatever shape is desired, embedded in a shape that matches the anatomy of the limb and fits perfectly into the window opening. This design will allow multiple iterations of the spring for different uses.

In another embodiment, the distal end 204 of the socket 100 (where the tip of the residual limb is supported) may be the location of the opening. The contour of the limb end may be converted to a filler 202 that fits above hardware that is mounted on the distal end 204 that connects the socket to the rest of the prosthesis.

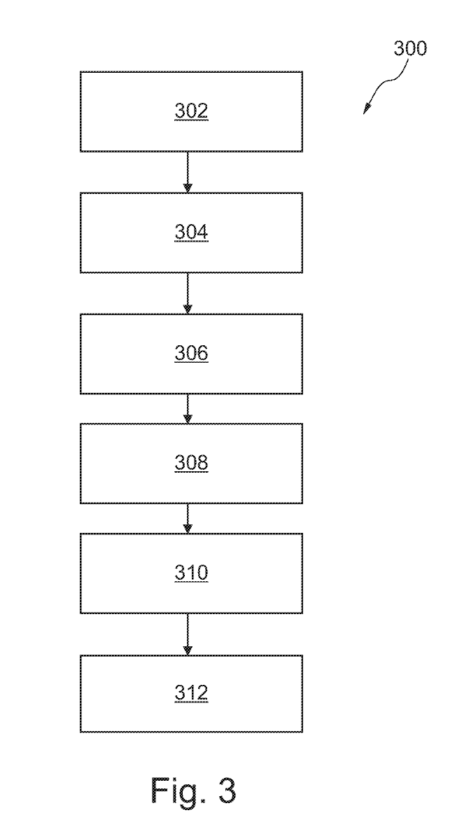

Referring now to FIG. 3, a process flow diagram of a method for changing local interface support geometry and materials within a prosthetic socket, according to an embodiment of the present disclosure, is shown. The following procedure is an embodiment for the process 300 of creating the socket:

1. Scan the target body part 302.

2. Convert the scan into a solid model using software known to those in the field 304.

3. Hollow out the virtual solid model leaving a thin wall that mimics the surface of the body part and is thickened away from the scanned surface 306.

4. Create the opening per the instructions provided by the prosthetist and the filler piece that matches the shape of the removed opening 308.

5. Insert the design feature, e.g. spring, at the location determined by the caregiver 310.

6. 3D Print the socket and filler piece 312.

A multitude of support and relief methods may be created around the shell of the socket using the process above.

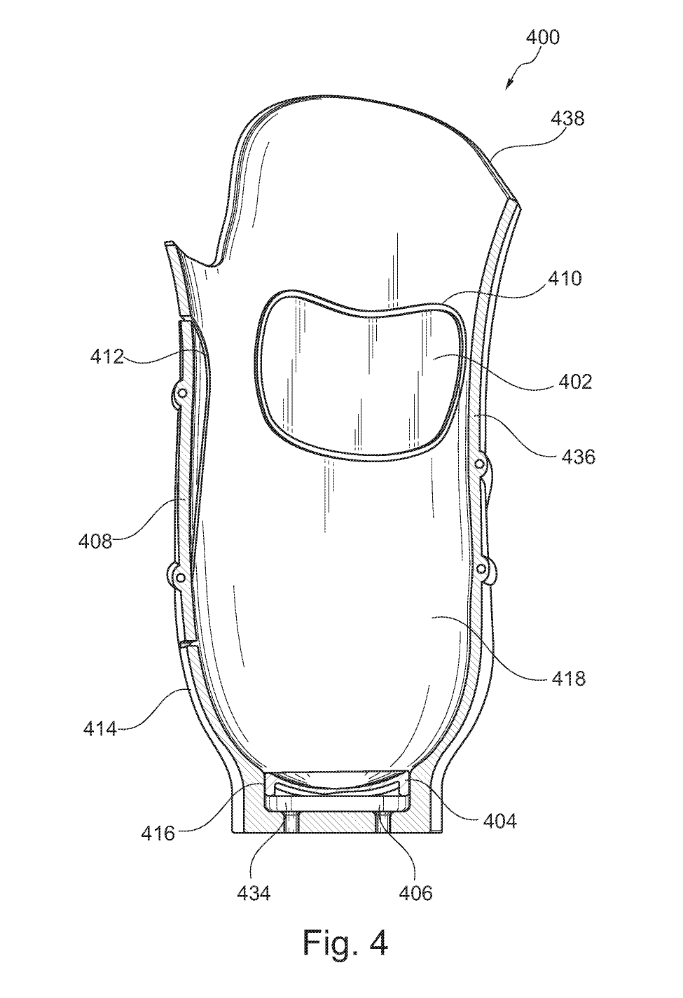

Referring now to FIG. 4, a plan view of a lower limb prosthetic socket 400 is shown. According to an embodiment of the present disclosure, lower limb prosthetic socket 400 is generally comprised of a medial interface panel 402, a distal cup 404, a distal interface support 406, a posterior interface panel 408, a medial panel window 410, a posterior panel window 412, an exterior socket surface 414, a distal interface portion 416, an interior socket surface 418, and at least one sensor 434. Lower limb prosthetic socket 400 is configured to enable a user of the socket to configure alternative support, pressure, and comfort characteristics of lower limb prosthetic socket 400 by selectively replacing medial interface panel 402, distal cup 404, and/or posterior interface panel 408 with an alternative medial interface panel 402, distal cup 404, and/or posterior interface panel 408 being configured to have different physical characteristics. Specifically, a user of lower limb prosthetic socket 400 may prefer a different “fit” for comfort and performance purposes when wearing lower limb prosthetic socket 400 in the context of different activities. For example, when a user of lower limb prosthetic socket 400 is engaging in higher intensity physical activity (e.g., exercising, jogging, biking, etc.), the user may prefer a tighter fit of lower limb prosthetic socket 400 on the user's residual lower limb. Conversely, when a user of lower limb prosthetic socket 400 is engaging in lower intensity physical activity (e.g., working in an office environment, engaging in personal activities, etc.), the user may prefer a more relaxed fit of lower limb prosthetic socket 400 on the user's residual lower limb.

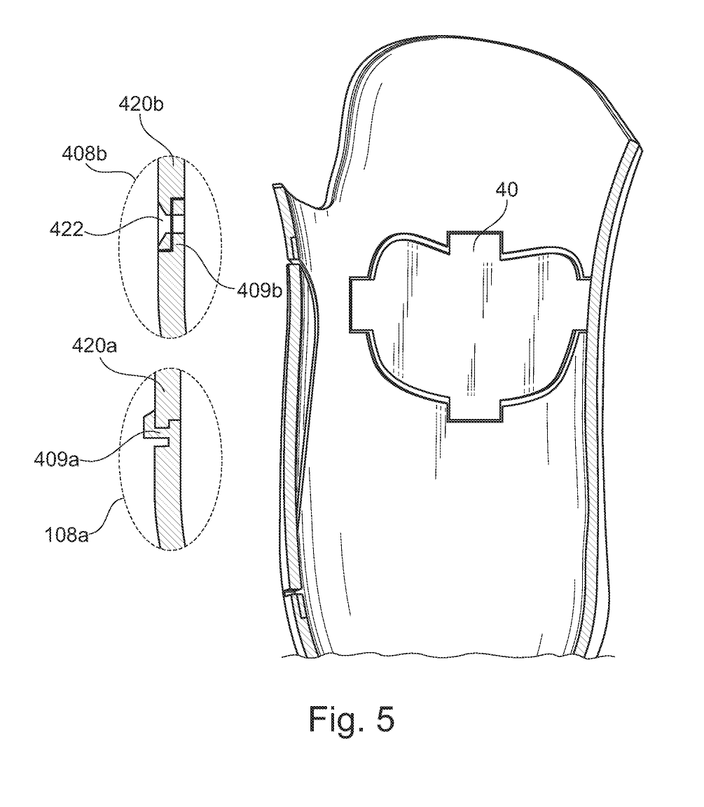

According to embodiments of the present disclosure, and still referring to FIG. 4, lower limb prosthetic socket 400 is configured to enable a variety of physical interface characteristics between lower limb prosthetic socket 400 and a user's residual lower limb through the dynamic configuration of medial interface panel 402, distal cup 404, and/or posterior interface panel 408. Medial interface panel 402 and medial panel window 410 are configured to provide interface support to a medial portion of the user's residual lower limb (e.g., the user's inner thigh). While not shown in FIG. 4, lower limb prosthetic socket 400 may further comprise a lateral interface panel and lateral panel window configured to provide interface support to a lateral portion of the user's residual lower limb (e.g., the user's outer thigh). Medial panel window 410 is configured to extend through the socket wall 436 to define a medial opening disposed on lower limb prosthetic socket 400. Medial panel window 410 may be configured in a variety of shapes, but should preferably be configured to be disposed adjacent to an upper medial portion of the user's residual lower limb when lower limb prosthetic socket 400 is worn. Medial interface panel 402 is selectively coupled to medial panel window 410 (as shown and described in more detail in FIG. 5, below). Medial interface panel 402 may be configured to have a complementary shape to that of medial panel window 410, such that medial interface panel 402 may fit within the boundaries of medial panel window 410. However, medial interface panel 402 may be configured in a variety of alternative shapes and geometries. For example, medial interface panel 402 as shown in FIG. 5 may be configured to be shorter in height than of medial panel window 410, thereby creating an exposed area at a lower or upper portion of medial panel window 410 when medial interface panel 402 is selectively coupled to medial panel window 410. Likewise, medial interface panel 402 may be configured to have a variety of apertures, cutouts, channels, and the like that create a variety of exposed areas (i.e. pass through portions) when medial interface panel 402 is selectively coupled to medial panel window 410.

Posterior interface panel 408 and posterior panel window 412 are configured to provide interface support to a posterior portion of the user's residual lower limb (e.g., the back of the user's thigh). While not shown in FIG. 4, lower limb prosthetic socket 400 may further comprise an anterior interface panel and anterior panel window configured to provide interface support to an anterior portion of the user's residual lower limb (e.g., the front of the user's thigh). Posterior panel window 412 is configured to extend through the socket wall 436 to define a posterior opening disposed on lower limb prosthetic socket 400. Posterior panel window 412 may be configured in a variety of shapes, but should preferably be configured to be disposed adjacent to a posterior portion of the user's residual lower limb when lower limb prosthetic socket 400 is worn. Posterior interface panel 408 is selectively coupled to posterior panel window 412 (as shown and described in more detail in FIG. 5, below). Posterior interface panel 408 may be configured to have a complementary shape to that of posterior panel window 412, such that posterior interface panel 408 may fit within the boundaries of posterior panel window 412. However, posterior interface panel 408 may be configured in a variety of alternative shapes and geometries. For example, posterior interface panel 408 as shown in FIG. 5 may be configured to be shorter in height than posterior panel window 412, thereby creating an exposed area at a lower or upper portion of posterior panel window 412 when posterior interface panel 408 is selectively coupled to posterior panel window 412. Likewise, posterior interface panel 408 may be configured to have a variety of apertures, cutouts, channels, and the like that create a variety of exposed areas (i.e. pass through portions) when posterior interface panel 408 is selectively coupled to posterior panel window 412.

Interior surface 418 of lower limb prosthetic socket 400 extends from an upper perimeter 438 to distal interface portion 416. Distal interface portion 416 is configured to receive and contain distal interface support 406 and distal cup 404. Distal interface support 406 may be configured at different heights according to the desired configuration of distal cup 404. Distal cup 404 is configured to provide interface support to the distal end of the user's lower residual limb during use of lower limb prosthetic socket 400. The size and shape of distal cup 404 may be configured to match the anatomy of the distal end of the user's lower residual limb by manufacturing distal cup 404 according to a digital scan of the user's lower residual limb. Distal cup 404 may be configured to enable a variety of comfort characteristics according to the desired use case. For example, and as described above, when a user of lower limb prosthetic socket 400 is engaging in higher intensity physical activity (e.g., exercising, jogging, biking, etc.), the user may prefer distal cup 404 to be configured to provide better dissipation of heat or have certain elasticity and porosity. Likewise, when a user of lower limb prosthetic socket 400 is engaging in lower intensity physical activity (e.g., working in an office environment, engaging in personal activities, etc.), the user may prefer distal cup 404 to be configured to be softer, smoother, or constructed of more comfortable material(s).

Distal cup 404 is configured to be housed in distal interface portion 416. Distal interface support 406 is configured to provide support to distal cup 404 such that the upper surface of distal cup 404 is properly aligned with interior surface 418 to match the contours of the lower residual limb of the user. Distal cup 404 may be constructed from a variety of materials, depending on the desired physical characteristics for distal cup 404: for example, rubber, plastic, silicone, and various composite and polymeric materials, and the like. Distal cup 404 may also have a variety of complementary components disposed on, or otherwise coupled to, distal cup 404 to help provide the desired comfort and performance characteristics (such as padding, covers, and the like).

At least one optional sensor 434 may be disposed in an interior portion of lower limb prosthetic socket 400. Sensor 434 may be disposed in distal interface portion 416; on a surface portion of interior surface 418; within socket wall 436; and/or on a bottom portion of distal cup 404. Sensor 434 may include one or more pressure sensors (such as a silicon piezoresistive pressure sensor); one or more temperature sensors (such as a silicon bandgap temperature sensor); one or more humidity sensors (such as a capacitive type humidity sensor); one or more motion sensors (such as an accelerometer, gyroscope, or ecompass); and/or, one or more electric impulse sensors (i.e. electrodes). Sensors 434 may work individually or in concert with two or more sensors 434 to measure physical characteristics of the interior portion of lower limb prosthetic socket 400, such as pressure, temperature, humidity, and movement/orientation of the residual lower limb. Sensors 434 may function to communicate sensor data to an electronic device of a user, and/or may function to send command data to one or more integrated electronic structures, such as an integrated cooling fan or a heat sink.

Referring now to FIG. 5, a plan view of lower limb prosthetic socket 400 and cross sectional views of posterior interface panel 408 and posterior panel window 412 are shown. According to embodiments of the present disclosure, posterior interface panel 408a may be selectively coupled to posterior panel window 412 by mechanically coupling a panel connector portion 409a with a panel window connector portion 420a. In this embodiment, panel connector portion 409a is “snap fit” with panel window connector portion 420a, such that posterior interface panel 408a may be selectively coupled to posterior panel window 412 without the use of tools or secondary fasteners. In another embodiment, posterior interface panel 408b is selectively coupled to posterior panel window 412 by coupling a panel connector portion 409b with a panel window connector portion 420b using a panel screw 422. While FIG. 5 illustrates two exemplary connection methods for posterior interface panel 408 and posterior panel window 412, the same connection methods shown in FIG. 5 are applied to medial interface panel 402 and medial panel window 410 (shown in FIG. 4). In addition to the mechanical coupling structures shown in FIG. 5, lower limb prosthetic socket 400 may also comprise one or more tensioning systems operably engaged with posterior interface panel 408 and posterior panel window 412, such as tensionable cables, straps, buckles, and the like.

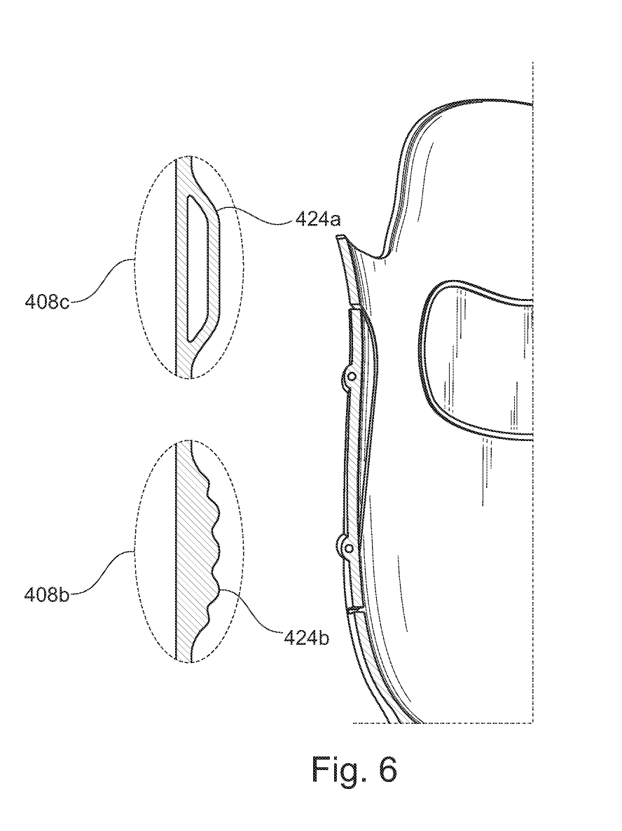

Referring now to FIG. 6, a plan view of lower limb prosthetic socket 400 and cross sectional views of alternative surface structures of posterior interface panel 408 are shown. According to an embodiment of the present disclosure, an interior surface 424 of posterior interface panel 408 may be configured according to predetermined performance characteristics. For example, interior surface 424a of posterior interface panel 408c may have a hollow interior structure, such that interior surface 424a of posterior interface panel 408c is compressible when coming into contact with the residual lower limb of a user. Alternatively, interior surface 424b of posterior interface panel 408d may have a solid interior structure and a corrugated surface configuration, such that interior surface 424b of posterior interface panel 408d maintains airgaps for ventilation when in contact with the residual lower limb of a user. As described above, a user may select to install posterior interface panel 408c or posterior interface panel 408d depending on the interface characteristics desired by the user, according to the user's use case. For example, the user may install interface panel 408c when greater comfort is desired (e.g. during low intensity activities), and may install interface panel 408d when greater performance and/or control is desired (e.g. during higher intensity activities).

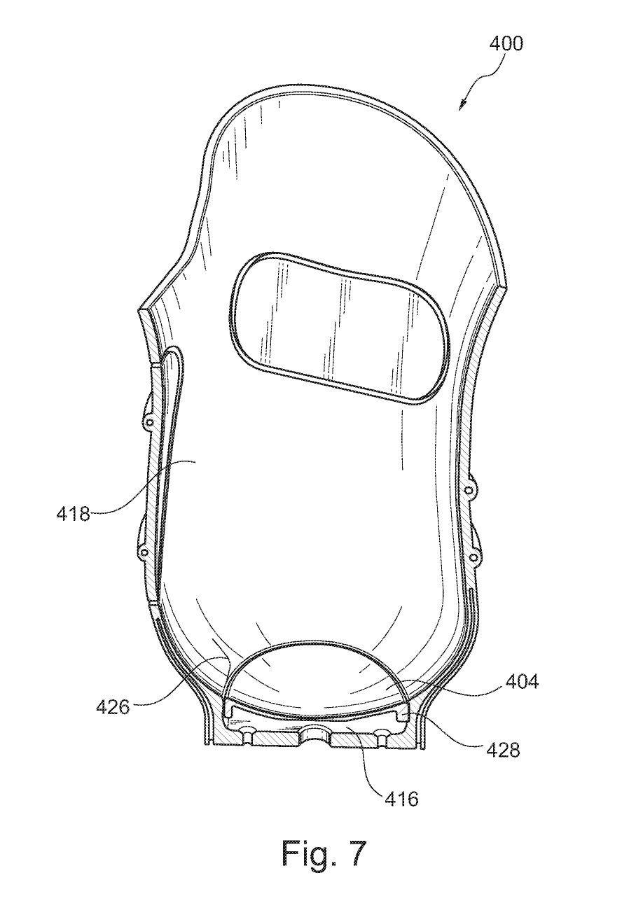

Referring now to FIG. 7, a plan view of lower limb prosthetic socket 400 is shown. According to an embodiment of the present disclosure, distal interface portion 416 is configured to receive and contain distal cup 404. A keyed channel 426 is disposed on interior portion 418 adjacent to distal interface portion 416. Keyed channel 426 is configured to guide distal cup 404 into its proper placement in distal interface portion 416. A distal cup guide key 428 is disposed on distal cup 404, and is configured to interface with a keyed portion of keyed channel 426 such that distal cup 404 maintains the proper orientation inside distal interface portion 416.

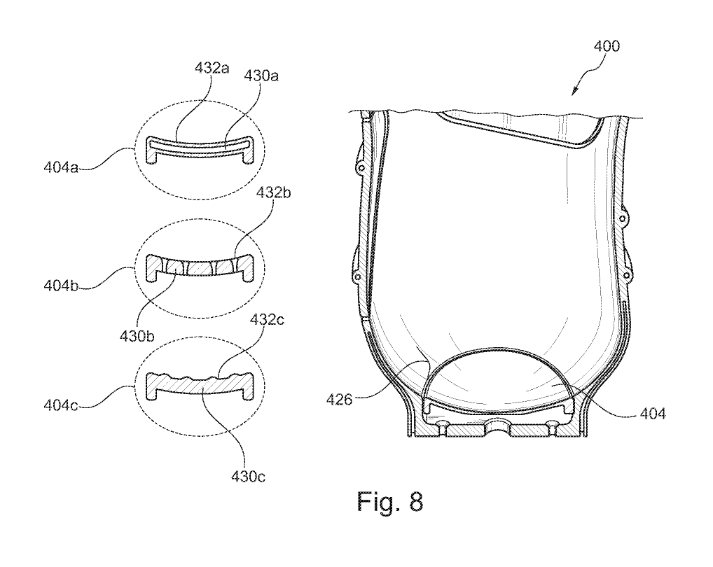

Referring now to FIG. 8, a plan view of lower limb prosthetic socket 400 and alternative cross sectional views of distal cup 404 are shown. As discussed in FIG. 4 above, the size and shape of distal cup 404 may be configured to match the anatomy of the distal end of the user's lower residual limb by manufacturing distal cup 404 according to a digital scan of the user's lower residual limb, and distal cup 404 may be configured to enable a variety of comfort characteristics according to the desired use case. According to embodiments of the present disclosure, distal cup 404a has a smooth upper surface 432a and a hollow interior structure 430a such that upper surface 432a of distal cup 404a is compressible when pressure is exerted on it by the distal end of the residual lower limb of a user. In another embodiment, distal cup 404b has an upper surface 432b with a plurality of venting structures disposed thereon and extending through interior structure 430b, such that distal cup 404b is better configured to dissipate heat from lower limb prosthetic socket 400. In yet another embodiment, distal cup 404c has a corrugated upper surface 432c and a solid interior structure 430c such that upper surface 432c maintains air gaps for ventilation when in contact with the distal end of a residual lower limb of a user. As with the interchangeability of the interface panels described above, a user may select to install distal cup 404a, distal cup 404b, or distal cup 404c depending on the interface characteristics desired by the user based on the user's use case. For example, the user may install distal cup 404a when greater comfort is desired (e.g. during low intensity activities), and may install distal cup 404c when greater performance and/or control is desired (e.g. during higher intensity activities).

The foregoing has outlined rather broadly the more pertinent and important features of the present invention so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present invention. It should be realized by those skilled in the art that such equivalent structures do not depart from the spirit and scope of the invention as set forth in the appended claims.

The present disclosure includes that contained in the appended claims as well as that of the foregoing description. Although this invention has been described in its exemplary forms with a certain degree of particularity, it is understood that the present disclosure of has been made only by way of example and numerous changes in the details of construction and combination and arrangement of parts may be employed without departing from the spirit and scope of the invention.

What is claimed is:

1. A modular lower-limb prosthetic apparatus comprising:
a 3D printed socket comprising an interior portion and an exterior portion defining a socket wall therebetween, the socket wall extending from an upper perimeter, defining a proximal opening of the interior portion, to a lower perimeter defining a distal plane, the distal plane defining a circumference, the 3D printed socket being configured according to a digital scan of a residual lower limb such that an interior surface of the interior portion mimics a surface of the residual lower limb, the 3D printed socket being constructed from a plurality of stratified layers;
a panel window being disposed on the 3D printed socket, the panel window comprising side walls extending from the interior portion of the 3D printed socket to the exterior portion of the 3D printed socket to define an aperture through the socket wall, the panel window having at least one panel interface portion being disposed on at least one of the side walls;
a first interface panel being removably coupled to the panel window, the first interface panel having at least one panel window interface portion being removably engaged with the at least one panel interface portion; and,
a first distal cup being removably coupled to a distal interface portion of the 3D printed socket, the first distal cup having an upper surface configured to mimic a distal surface of a residual limb of a user, and a lower surface configured to interface with the distal interface portion of the 3D printed socket.

2. The modular lower-limb prosthetic apparatus of claim 1 further comprising a second interface panel being configured to be removably coupled to the panel window in place of the first interface panel, the second interface panel having at least one panel window interface portion being removably engaged with the at least one panel interface portion.

3. The modular lower-limb prosthetic apparatus of claim 1 further comprising a second distal cup being configured to be removably coupled to a distal interface portion of the 3D printed socket in place of the first distal cup, the second distal cup having an upper surface configured to mimic a distal surface of a residual limb of a user, and a lower surface configured to interface with the distal interface portion of the 3D printed socket.

4. The modular lower-limb prosthetic apparatus of claim 1 further comprising at least one sensor disposed on a surface of the interior portion of the 3D printed socket, the at least one sensor being operably configured to measure one or more characteristics of the interior portion of the 3D printed socket when being worn by the user.

5. The modular lower-limb prosthetic apparatus of claim 1 wherein the first distal cup and the distal interface portion of the 3D printed socket are configured to be mateably coupled such that the first distal cup may be manually installed and removed from the distal interface portion of the 3D printed socket by the user.

6. The modular lower-limb prosthetic apparatus of claim 2 wherein one or more support, pressure, and comfort characteristics of the 3D printed socket are selectively configured by removably coupling the second interface panel to the panel window in place of the first interface panel.

7. The modular lower-limb prosthetic apparatus of claim 2 wherein the first interface panel and the second interface panel are configured to differ in shape.

8. The modular lower-limb prosthetic apparatus of claim 2 wherein the first interface panel and the second interface panel are operably configured to exert differing pressure against the interior portion of the 3D printed socket relative to each other.

9. The modular lower-limb prosthetic apparatus of claim 3 wherein at least the upper surface of the first distal cup and the upper surface of the second distal cup at least are configured to differ in hardness or porosity relative to each other.

10. A variable interface lower-limb prosthetic socket apparatus comprising:
a 3D printed socket comprising an interior portion and an exterior portion defining a socket wall therebetween, the socket wall extending from an upper perimeter, defining a proximal opening of the interior portion, to a lower perimeter defining a distal plane, the distal plane defining a circumference, the 3D printed socket being configured according to a digital scan of a residual lower limb such that an interior surface of the interior portion mimics a surface of the residual lower limb, the 3D printed socket being constructed from a plurality of stratified layers;
a panel window being disposed on the 3D printed socket, the panel window comprising side walls extending from the interior portion of the 3D printed socket to the exterior portion of the 3D printed socket to define an aperture through the socket wall, the panel window having at least one panel interface portion;
at least one interface panel being removably coupled to the panel window via the at least one panel interface portion, the at least one interface panel being operably configured to define one or more support, pressure, and comfort characteristics of the 3D printed socket; and,
a distal cup being mateably coupled to a distal interface portion of the 3D printed socket such that the distal cup is configured to be selectively installed or removed from the distal interface portion of the 3D printed socket by the user, the distal cup having an upper surface configured to mimic a distal surface of a residual limb of a user, and a lower surface configured to interface with the distal interface portion of the 3D printed socket.

11. The variable interface lower-limb prosthetic apparatus of claim 10 further comprising at least one sensor disposed on a surface of the interior portion of the 3D printed socket, the at least one sensor being operably configured to measure one or more characteristics of the interior portion of the 3D printed socket when being worn by the user.

12. The variable interface lower-limb prosthetic apparatus of claim 10 further comprising a variable tensioning means operably engaged with the at least one interface panel.

13. The variable interface lower-limb prosthetic apparatus of claim 10 further comprising a comfort material removably disposed on the interior portion of the 3D printed socket.

14. The variable interface lower-limb prosthetic apparatus of claim 10 further comprising a cooling means operably engaged with the interior portion of the 3D printed socket.

15. The variable interface lower-limb prosthetic apparatus of claim 10 wherein the upper surface of the distal cup further comprises one or more interchangeable comfort components.

16. The variable interface lower-limb prosthetic apparatus of claim 11 wherein the at least one sensor is selected from the group consisting of pressure sensors, temperature sensors, humidity sensors, motion sensors, and electric impulse sensors.

17. A modular lower-limb prosthetic system for interchangeable local interface support, the modular lower-limb prosthetic system comprising:
a 3D printed socket comprising an interior portion and an exterior portion defining a socket wall therebetween, the socket wall extending from an upper perimeter, defining a proximal opening of the interior portion, to a lower perimeter defining a distal plane, the distal plane defining a circumference, the 3D printed socket being configured according to a digital scan of a residual lower limb such that the interior surface of the socket wall mimics a surface of the residual lower limb, the 3D printed socket being constructed from a plurality of stratified layers;
a panel window being disposed on the 3D printed socket, the panel window comprising side walls extending from the interior portion of the 3D printed socket to the exterior portion of the 3D printed socket to define an aperture through the socket wall, the panel window having at least one panel interface portion being disposed at least one of the side walls;
a first interface panel being removably coupled to the panel window, the first interface panel having at least one panel window interface portion being removably engaged with the at least one panel interface portion;
a first distal cup being removably coupled to a distal interface portion of the 3D printed socket, the first distal cup having an upper surface configured to mimic a distal surface of a residual limb of a user, and a lower surface configured to interface with the distal interface portion of the 3D printed socket;
a second interface panel being configured to be removably coupled to the panel window in place of the first interface panel, the second interface panel having at least one panel window interface portion being removably engaged with the at least one panel interface portion; and,
a second distal cup being configured to be removably coupled to a distal interface portion of the 3D printed socket in place of the first distal cup, the second distal cup having an upper surface configured to mimic a distal surface of a residual limb of a user, and a lower surface configured to interface with the distal interface portion of the 3D printed socket.

18. The modular lower-limb prosthetic system of claim 17 further comprising at least one sensor disposed on a surface of the interior portion of the 3D printed socket, the at least one sensor being operably configured to measure one or more characteristics of the interior portion of the 3D printed socket when being worn by the user.

19. The modular lower-limb prosthetic system of claim 17 wherein the first interface panel and the second interface panel are operably configured to exert differing pressure against the interior portion of the 3D printed socket relative to each other.

20. The modular lower-limb prosthetic system of claim 17 wherein at least the upper surface of the first distal cup and the upper surface of the second distal cup at least are configured to differ in hardness or porosity relative to each other.
